(19) United States
(12) Patent Application Publication
(10) Pub. No.: US 2002/0138139 A1
(43) Pub. Date: Sep. 26, 2002

(54) Presbyopia treatment by scleral compression

(76) Inventor: Jonathan S. Till, Salem, VA (US)

Correspondence Address: KENYON & KENYON, 1500 K STREET, N.W., SUITE 700, WASHINGTON, DC, 20005, US

(21) Appl. No.: 10/102,980

(22) Filed: Mar. 22, 2002

Related U.S. Application Data
(60) Provisional application No. 60/277,626, filed on Mar. 22, 2001.

(51) Int. Cl. A61F 2/14

(52) U.S. Cl. 623/4.1; 623/905; 128/898

(57) ABSTRACT

The invention discloses methods and apparatus that reverse or eliminate presbyopia by affecting a change in the shape of the sclera. The change can be designed to reduce tension placed on the lens from the zonules, thereby allowing the lens to bulge about its central axis.

The present application claims priority to the provisional application No. 60/277,626 filed Mar. 22, 2001 by the inventor named herein.

### BACKGROUND DISCUSSION

Presbyopia affects virtually every person over the age of 44. According to Jobson Optical Database, 93% of people 45 and over are presbyopic. Presbyopia entails the progressive loss of amplitude of accommodation that occurs with aging. Adler's Physiology of the Eye, which is incorporated herein by reference for background information, discloses that the human accommodative amplitude declines with age such that accommodation is substantially eliminated by the age of 50 to 55. Accommodative ability has been defined as the capacity of the eye to focus for near vision by changing the shape of the lens to become more convex.

The accommodative amplitude of the lens is measured in diopters (D). It has been documented that the loss of accommodative ability begins at a very early age, such that by age ten the average eye has 10 D, age thirty, 5D, and by age forty, only 2.5D of accommodative power. The lens of a person who does not suffer from presbyopia (i.e., a person whose lens accommodates normally), will typically have an accommodative amplitude of about 2.5 diopters or greater. The terms “reversing presbyopia” or “treating presbyopia” are often used to denote an increase in the range of focus of the lens, for example by creating additional focal points.

The ocular tissues involved in the accommodative response include the lens, the zonules, the lens capsule, and the ciliary muscle. Of these, the lens is the central tissue. These structures function together to enable the eye to focus on close objects by changing the shape of the lens. As will be discussed in greater detail, the lens is centrally suspended between the anterior and posterior chambers behind the pupillary opening of the iris. The lens is supported by an array of radially oriented zonular fibers, which extend from the lateral edges of the lens to the inner border of the circumferential ciliary muscle. The ciliary muscle is attached to the scleral coat of the eye. When the eye is at rest, it is focused for distance and the lens is in a somewhat flattened or less convex position. This shape is due to the tension that is exerted on the lens periphery by the zonules. The zonules pull the edges of the lens toward the ciliary body.

During accommodation, the shape of the lens becomes more convex through contraction of the ciliary muscle, which allows the ciliary attachment of the zonules to move toward the lens, reducing the tension in the anterior zonules. This reduction in tension allows the central region of the lens to increase in convexity, thereby enabling near objects to be imaged on the retina. The processes involving the coordinated effort of the lens, zonules, ciliary body, medial rectus muscles and iris, among others, that leads to the ability of the eyes to clearly focus near on the retina is known as the accommodative process.

Several theories have been advanced to explain the loss of accommodation with age. These theories include the hardening of the lens with age, loss of strength in the ciliary muscle, and, the loss of elasticity of the lens capsule. As for the loss of strength of the ciliary muscle, it is noted that although there are age-related morphological changes that occur, there is little evidence of the diminishing strength of the ciliary muscle. In fact, it has been shown that under the influence of pilocarpine, the ciliary muscle will vigorously contract even in presbyopic eyes.

As for changes in the lens capsule, it has been postulated that reduction in the elasticity of the capsule is, in fact, a contributing factor in presbyopia. Moreover, it has been found that Young's mode of elasticity for the lens capsule decreases by nearly 50% from youth to age 60, while accommodation decreases by 98%. Consequently, the principal cause of presbyopia is now considered to be “lenticular sclerosis” or the hardening of the lens.

A cataract is a condition in which the lens becomes less clear. The study of cataracts lends insight into lens and capsular changes. The usual senile cataract is relatively discus-shaped when removed from the eye, its shape being dictated by the firm lens substance. The liquefied hypermature cataract is globular when extracted, rounded up by the elastic lens capsule. This is indirect evidence that it may be possible to reverse the lenticular changes associated with presbyopia, and that the lens capsule may still be sufficiently elastic.

Other theories advanced to explain presbyopia involve the role of lens growth through life and the loss of tension on the lens capsule. These theories, however, have not been supported by clinical observations.

At the time of this invention, common treatments for presbyopia include reading glasses, bifocal glasses, or mono-vision contact lenses. All of these solutions necessitate the use of an appliance creating additional shortcomings. Alternative theories for treating presbyopia include scleral expansion and corneal reshaping. The efficacy of such techniques is not well established.

### BRIEF DESCRIPTION OF THE DRAWINGS

The various features of the invention will be appreciated by simultaneous reference to the description which follows and the accompanying drawings, wherein like numerals indicated like elements, and in which:

FIG. 1 is a schematic representation of a horizontal section of the eye;

FIG. 2A is a schematic representation of exemplary prosthetic device that can be used to advance the principles of the invention;

FIG. 2B is the cross sectional view of the embodiment illustrated in FIG. 2A;

FIG. 3A is a schematic representation showing the placement of prosthetic apparatus within the scera;

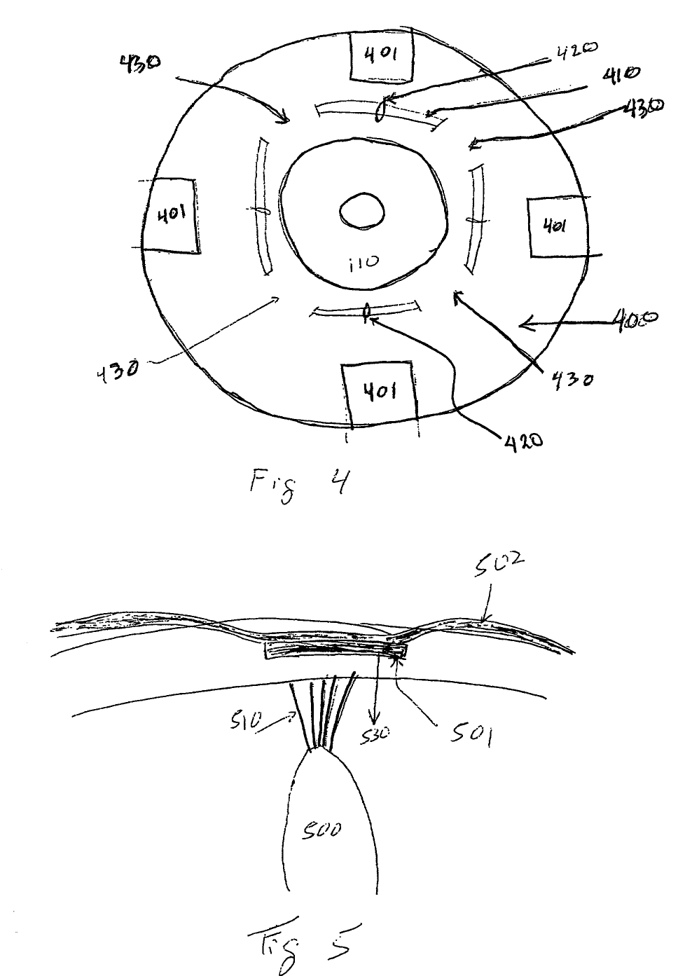

FIG. 2B is the cross sectional view of the embodiment illustrated in FIG. 3A;

FIG. 4 is a schematic representation of the bio-compatible prosthesis on the globe; and

FIG. 5 is a schematic representation showing a magnified view of a scleral tunnel with a band 502 and indentation 501 in place.

### DETAILED DESCRIPTION OF AN EMBODIMENT OF THE INVENTION

Presbyopia, or the loss of the accommodative amplitude of the lens, has often advanced in a typical person age 45 or older to the point where some type of corrective lens in the form of reading glasses or other treatment is required. It is to be understood that loss of accommodative amplitude can occur in persons much younger or older than the age of 45, thus the present invention is not to be construed as limited to the treatment of presbyopia in a person of any particular age. The present invention can be most useful in a person whose accommodative amplitude has lessened to a point where restoration thereof to some degree is desirable.

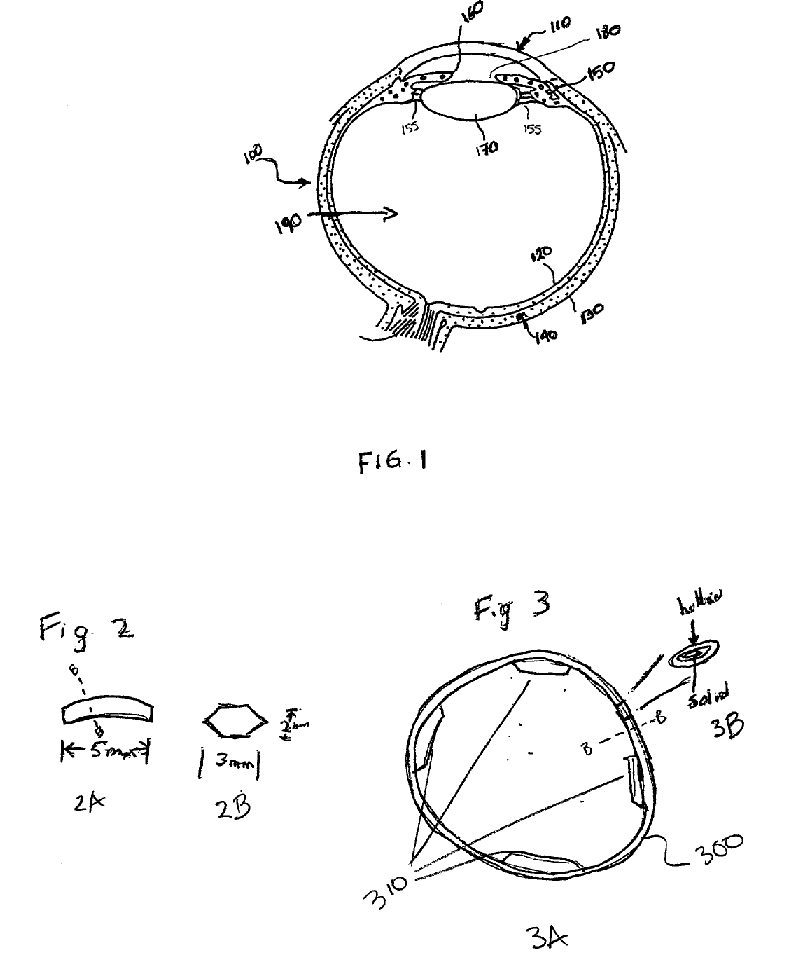

FIG. 1 is a schematic representation of a horizontal section of the eye. Referring to FIG. 1, globe 100 of the eye resembles a sphere with cornea 110 representing the bulging anterior of the eye. The retina 120 is concentric with globe 100 and includes a light sensitive region for capturing the light that passes through the lens. Sclera 130 is a fibrous portion and comprises the outermost covering of the globe. Cornea 110 forms the anterior portion of the globe and is transparent.

Choroid 140 is vascular and nutritive in nature and, among other things, maintains retina 120. Ciliary body 150 suspends lens 170 and provides the necessary movements of the lens to provide accommodation. Iris 160 is also arranged in the anterior portion of the eye and forms a circular disc similar to a diaphragm of a camera. Iris 160 includes the perforated central region known as pupil 180. In addition to controlling the size of pupil 180 to expose retina to light, iris 160 contracts with accommodation.

The area between iris 160 and cornea 110 is the anterior chamber and the area between iris 160 and retina 120 is called the posterior chamber. Posterior chamber 190 is a vitreous and gelatinous mass filling the posterior portions of globe 100, supporting at its sides ciliary body 150 and retina 120 and housing lens 170.

As shown in FIG. 1, lens 170 is placed between iris 160 and vitreous body 190. As lens 170 accommodates its diameter changes. Zonules 155 are transparent fibers interposed between ciliary body 150 and lens 170. In essence, zonules 155 hold lens 170 in position allowing the ciliary muscle to act upon the curvature of the lens. Thus, contraction of ciliary muscle causes the ciliary body 150 to move towards the equator or the center of lens 170. This causes zonules 155 to relax their tension on lens 170, thereby allowing lens 170 to assume a more spherical shape. During accommodation, the main change is in the central radius of curvature (i.e., the central regions) of the anterior lens surface. By way of example, the anterior lens surface having a radius of 12 mm in the unaccommodative state can have a radius of 3 mm during accommodation. Both the peripheral anterior and the posterior lens surfaces change very little in curvature during accommodation. The axial thickness of lens 170 increases while the diameter thereof decreases. In other words, during accommodation lens 170 bulges. This bulge is more pronounced in the central regions than in the periphery of the lens capsule 170. The central anterior lens capsule is thinner than the rest of the anterior capsule. This may explain why lens 170 bulges more centrally during accommodation. The thinnest portion of the capsule is the posterior capsule, which has a curvature greater than the anterior capsule in the unaccommodative state. The protein content of the lens, almost 33% by weight, is higher than any other organ in the body.

To improve accommodative ability of the lens, in one embodiment, the present invention contemplates a method and apparatus for compensating, reversing or eliminating the effects of presbyopia through induction of a double astigmatic optical distortion of the lens by way of applied extra-ocular pressure to thereby effect scleral compression in the region of the zonular attachments of the lens.

Astigmatic optical distortion can be considered as providing force in the form of tension or compression to the region of sclera in order to change the dioptic power along at least one axis of the lens. Similarly, extra-ocular pressure to the sclera can cause a graduated change in dioptic power along two axes to create a double astigmatic optical distortion. It will be understood that the inventive principles provided herein can be applied in different directions and/or in multiple axis.

An extra-ocular pressure can be defined as an inwardly directed force which causes a change in the dioptric power of the lens in the region of the lens at which the external force has been applied. The force can be applied upon the sclera and translated through the tissue to the zonules which causes a relaxation of the zonules in the region. This then causes a diminution of force being applied the lens. The extra-ocular force can be radially directed.

By applying extra-ocular pressure to the sclera along two different axes, a graduated change in dioptic power along these two axes will be affected thereby creating a multifocal lens system. If these two axes are placed 90 degrees apart from each other, then the net effect will be two gradients of induced regular astigmatism directly perpendicular to each other. This optical arrangement, although resulting in optical distortions, can generate a multifocal imaging system much like those using concentric rings of varying power as found in multifocal contact lenses or the gradient found in graduated bifocal lenses. As stated, the principles of this invention are not limited to applying forces along only two axes. Indeed, the principles of the invention can be applied to a system wherein multiple distortions are introduced along one or more axes to produce the desired net result. Thus, in one embodiment, the present invention provides a novel approach to reversing the effects of presbyopia by inducing certain changes in the optical properties of the lens, which afford a greater range of focus. These changes can involve the creation of a double astigmatic error with perpendicular axes. As will be discussed in greater detail, creating of multiple focal points can be implemented according to the various embodiments of the invention by, for example, inserting a bio-compatible prosthesis into the sclera, by inserting an annular-type band having various indentations thereon into the sclera or by reshaping the sclera by applying energy in order to create the inwardly directed pressure. While these embodiments can induce certain levels of distortion to the lens, the distortions can enable the patient to have both near and distant vision without the need for glasses. Further, while not wishing to be bound to any specific theory, the distortion can be observed mainly in the lens and can create the multi-focal effect needed to combat presbyopia.

Thus, in one embodiment, the present invention is directed to a method for reversing and/or treating presbyopia by use of an extra-ocular bio-compatible prosthetic device, which can induce a certain amount of inward compression on the eye in the area of the ciliary muscle.

Exemplary embodiments of the prosthesis can include two general categories: the prosthesis can comprise one or more segment, or alternatively, the prosthesis can be a unitary device. The prosthetic segments are usually made from any bio-compatible material in general, and bio-compatible semi-pliable plastic material in particular, and can be either sewn to the sclera or imbedded into the sclera after the creation of scleral tunnels. Creation of the scleral tunnels are conventionally known to an ordinary skilled artisan. These prosthetic segments, for example, can be 5 to 10 mm in length, 2-4 mm thick and 3-5 mm wide. Further, the bio-compatible prosthesis can have any shape that provides the desired force. For example, the prosthetic device can be hexagonal, rectangular or circular in cross section. Again, it should be noted that the specific dimensions and geometries provided herein are exemplary, and should not be construed to limit the scope of the invention.

FIG. 2 is a schematic representation of exemplary prosthetic device that can be used to advance the principles of the invention. Specifically, FIG. 2A shows a segment of an annular prosthesis, while FIG. 2B shows the cross sectional view of one exemplary prosthesis. In addition to the annular, or ring-shaped prosthesis, one ore more wedges (not shown) can be inserted at appropriate locations of the sclera in order to produce the desired multiple distortions. Such wedges can have any that lends itself to creating astigmatic error.

Another exemplary form of a bio-compatible prosthesis can include an torroidal or elliptical band or an open ring. In one embodiment, the annular prosthesis band include four evenly spaced indentations or protrusions along the inner aspect of the band.

FIG. 3 is a schematic representation showing the placement of prosthetic apparatus within the sclera. Referring to FIG. 3A, annular band 300 is shown to have protrusions 310 which cause corresponding indentations on the sclera. The cross sectional view of band 300 is shown in FIG. 3B. The exemplary embodiment of FIG. 3B illustrates a hollow band. It is noted that departure from the exemplary embodiments and geometries of the prosthetic devices represented herein will be well within the scope of the invention.

By way of example, protrusions 310 can be 5-10 mm long. Band 300 can be a flexible plastic such as silastic with one end hollow and the other end solid with a smaller diameter such that the smaller end and can be introduced into the hollow end or threaded into the hollow end to varying depth to allow control on the amount of external tension that is placed upon the sclera. Band 300 can be oval rather or perfectly round in cross section. The length of the prosthetic device can be adjusted at time of the surgery after the band has been applied to globe 100 (FIG. 1) in the region of the ciliary body.

The prosthetic device can be constructed from conventional bio-compatible material, and preferably from material suited for the implanting into the eye. Common examples of suitable material are disclosed in U.S. Pat. No. 6,214,044 to Silverstrini, which is incorporated herein for background information.

FIG. 4 is a schematic representation of the bio-compatible prosthesis on the globe. Referring to FIG. 4, band 410 can be sewn to globe 400 using non-absorbable sutures 420 and in cooperation with scleral tunnels 430 made large enough to thread the band through sideways. For example, tunnels 430 can be made along the 45 meridians. Tunnels 430 are preferably not made front of the rectus muscles insertions 401. After band 410 is threaded through the four scleral tunnels 430, it can be rotated with the protrusions being contained within the tunnels to obtain the desired multi-focal effect. Thereafter, band 410 can be sewn to the sclera in the regions between each of the indentations. While the exemplary embodiments presented herein are directed to implanting the prosthesis within the sclera, it will be noted that invention should not limited thereto. For example, the prosthesis can be brought to intimate contact with the sclera, without penetrating thereto, in order to bring about the astigmatic distortion contemplated herein.

FIG. 5 is a schematic representation showing a magnified view of a scleral tunnel with band 502 and indentation 501 in place. Protrusions 501 can be in a position to create inwardly directed pressure upon zonules 510 and affect relaxation, or a reduction, of tension on lens 500 allowing lens 500 to assume a greater curvature along this axis. In the schematic representation of FIG. 5, direction of the force is represented by arrow 530.

In still another embodiment of the invention, energy can be applied to the outside wall of the eye to induce shrinkage of the sclera thereby affecting regional scleral compression. According to this embodiment, external energy can be used to create regional indentation of the sclera. Such indentations can force the sclera to shrink relative to its original size. The shrinkage of sclera can lead to relaxing the tension on the zonules, consequently allowing lens 50 to assume a greater curvature.

Particularly, this and other embodiments of the invention can affect a change in the range of focus of the human lens by, for example: (1) inducing a distortion to the lens by way of an externally applied prosthetic device which allows for multiple depths of focus including near, and/or (2) the use of externally applied energy to affect a change in the range of focus of the human lens through affecting a shrinkage of the sclera and thereby creating an indentation of the sclera and a relaxation of the zonules. As can be seen, in this embodiment of the invention, the change in the physical geometry of the sclera translates directly to the lens. For example, affecting a physical change in the geometry of the sclera, relaxes the tension placed on the lens from the zonules and enables further bulging of the lens. It can also be seen that as a result of the physical change to the sclera, additional distortions are introduced, creating multiple focal points and enabling the presbyopic eye to have a better range of focus. While not wishing to be bound to any specific theory, it is believed that by inducing these distortions to the lens, the invention has the effect of creating a multifocal optical system for the eye thereby compensating for the effects of presbyopia. In other words, as the presbyopic condition progresses, the focal point of the lens increasingly moves further away from the lens. By affecting the sclera to create, for example, four different distortion in the sclera, four new focal points can be created, making the lens multi-focal. Thus, the distortion greatly increases the lens' range by creating multiple focal points.

In one embodiment of the present invention, the treatment(s) contemplated herein can result in an increase in the range of focus of at least about 0.5 diopters. In another embodiment of the invention, the treatment(s) contemplated herein can result in an increase in the range of 0.2 to 0.8 diopters.

The embodiment of the invention can result in an increase in the range of focus because the change in the geometry of the sclera can create a distortion which counteracts the tendency toward a single focal point as caused by the effects of presbyopia. In addition, it is noted that the distortion introduced by the prosthetic device can compensate for the loss of elasticity of the lens.

In another embodiment of the present invention, the method of treating presbyopia can result in an increase in the range of focus of at least about 2.0 diopters. It is noted that while it can be most beneficial to restore the range of focus of the lens to normal, a lesser degree of restoration can be also beneficial. For example, in some cases advanced presbyopia can cause severe reduction in the range of focus, thus making a complete restoration improbable. In this and similar cases, the inventive embodiment of the invention can at least partially restore vision such that the use of appropriate lenses can overcome the presbyopic condition.

As stated, the inelasticity of the lens, or the hardening thereof, can be a contributing cause of presbyopia. The hardening of the lens can be due to an alteration of the structural proteins or an increased adhesion between the lens fibers. In one embodiment, the present invention is directed to treating presbyopia by inducing a distortion such as a double astigmatic error with substantially perpendicular axes. In another embodiment, the present invention contemplates treating presbyopia by inducing a double astigmatic error having angles that are not perpendicular. In yet another embodiment of the invention, presbyopia is treated by inducing a single astigmatic error which distorts the lens sufficiently to treat presbyopia. Although this may cause some distortion to the images, it will increase the range of focus and the depth of field.

In a double astigmatic error approach of the invention, the lens capsule can be distorted in two different directions. In an exemplary embodiment where the direction of astigmatic distortion is represented by an axis, the double astigmatic distortion would distort the lens in two different directions. The axes of the distortions can be substantially perpendicular to each other. In an alternative embodiment, the axes can be substantially non-perpendicular to each other. It would follow that the angle between the axis can be adjusted to accommodate each individual treatment. In this regard, FIG. 5 shows the direction of the force 530 as created can be created by indentation 501.

For example, in treating one patient, the best result may obtained by inducing a double astigmatic distortion wherein the distortion axes are substantially perpendicular to one another; while in treating another patient the axis can have an acute or obtuse angulation.

Thus, in one embodiment of the invention treatment process involves applying a scleral prosthesis to affect either regional or global compressive forces to the eye. As stated, this can be done with an annular band, or a segment thereof, with or without inwardly directed focal regions of additional material. Non-limiting examples of intrascleral implants can include bio-compatible hard plastic slivers, soft flexible plastic, viscous fluids, or other space occupying bio-compatible material.

In another embodiment of the invention, the treatment process involves applying energy to the sclera to affect a physical change in the geometry of the sclera. By way of example, the energy source can include: laser, ultrasound, heat, infrared, microwave, visible light, and other bio-compatible electromagnetic energy. For example, energy, as laser, can be used to affect a physical change in the shape of sclera. This method can result in a substantial prevention of the reoccurrence of presbyopia.

In yet another embodiment, the treatment process involves applying energy to the sclera in the region of the ciliary body. This energy causes the collagen in the sclera to shrink thereby creating an indentation of the sclera. While in embodiments using energy, do not require introducing a prosthesis, there may be occasions where both embodiments can be used in combination.

Thus, in one embodiment of the invention, the increase in the range of focus is accomplished by treating the sclera with radiation, ionizing, sonic or electromagnetic energy, heat, chemical, particle beam, plasma beam, enzyme, other energy source, and/or any combination of any of the above sufficient to cause the scleral wall to shrink or contract either in localized regions or globally. As examples, lasers useful in the present invention include: excimer, argon ion, krypton ion, carbon dioxide, helium-neon, helium-cadmium, xenon, nitrous oxide, iodine, holmium, yttrium lithium, dye, chemical, neodymium, erbium, ruby, titanium-sapphire, diode, any harmonically oscillating laser, or any other electromagnetic radiation. Exemplary forms of heating radiation include: infrared, heating, infrared laser, radiotherapy, or any other methods of heating the lens. Finally, exemplary forms of sound energy that can be used in an embodiment of the invention include: ultrasound, any audible and non-audible sound treatment, and any other biologically compatible sound energy. Such treatment can cause an indentation (or protrusion) of the sclera which would lead to relaxation of the zonules.

Chemicals that can cause the contraction of collagen would be the most useful agents for the induction of scleral contraction. Various bio-compatible caustic and alkylating agents can be used.

The external energy used with various embodiments and methods of the present invention could be applied through either contact with the sclera or non-contact techniques of delivery. More than one treatment may be needed to affect a suitable increase in the range of focus. When more than one modality of treatment is desirable, chemical treatment can be administered prior to, after, or simultaneously with the application of externally applied energy. Typically, the energy can be applied in the region of the ciliary body and for a predetermined amount of time based on the patient's age. While a number of sites can be targeted, in one embodiment a minimum of four sites can be chosen on the sclera. After anesthetizing the eye with a topically applied anesthetic agent, the treatment can be applied through the conjunctiva and into the sclera. The patient is then tested after, for example, one month and if the results are not adequate, then an additional treatment may be delivered using half the original exposure time.

The disclosed embodiments are illustrative of the various ways in which the present invention may be practiced. Other embodiments can be implemented by those skilled in the art without departing from the spirit and scope of the present invention. It is understood by one of ordinary skill in the art that the above-cited embodiments are exemplary in nature and other combinations, subcombination or variations embodying the principles of the invention are considered to be well within the scope of the claimed invention. Is should be understood that claimed invention may include other embodiments not specifically disclosed.

What is claimed is:

1. A method for treating presbyopia comprising affecting a change in the shape of an sclera of the eye, the change adapted to reduce tension placed on the lens from a zonules.

2. A method for treating presbyopia comprising inducing a double astigmatic error to the lens by reducing tension on a group of zonules supporting a lens.

3. An apparatus for treating presbyopia comprising a bio-compatible substrate for intimate contact with sclera, the substrate adapted to cause at least one astigmatic distortion to the lens.
